(19) United States
(12) Patent Application Publication
(10) Pub. No.: US 2023/0329907 A1
(43) Pub. Date: Oct. 19, 2023

(54) DIGITAL AWARENESS SYSTEM FOR OPHTHALMIC SURGERY

(71) Applicant: Alcon Inc., Fribourg (CH)

(72) Inventors: Lu Yin, Keller, TX (US); Kongfeng Berger, San Jose, CA (US); Ramesh Sarangapani, Coppell, TX (US); Vignesh Suresh, Woodland Hills, CA (US)

(21) Appl. No.: 18/299,022

(22) Filed: Apr. 11, 2023

Related U.S. Application Data
(60) Provisional application No. 63/332,106, filed on Apr. 18, 2022.

(51) Int. Cl. A61F 9/007; A61B 3/14; A61B 34/20

(52) U.S. Cl. CPC A61F 9/007; A61B 3/14; A61B 34/20; A61B 2090/372

(57) ABSTRACT

Certain embodiments provide a method of performing ophthalmic surgical procedures. The method includes ingesting and preparing pre-operative data and intra-operative data associated with a patient's eye for further processing. In certain embodiments, the method further includes integrating the pre-operative data and intra-operative data to generate context sensitive data for further processing. The method also includes classifying and annotating the pre-operative data, the intra-operative data, and the context sensitive data. The method also includes extracting one or more actionable inferences from the pre-operative data, the intra-operative data, context sensitive data, and the classified and annotated data. The method further includes triggering, based on the one or more actionable inferences, one or more actions on an imaging system or a surgical system.

### BACKGROUND

A variety of diseases or conditions associated with an eye may be treated through ophthalmic surgical procedures. Examples of ophthalmic surgical procedures include vitreo-retinal surgery, cataract surgery, glaucoma surgery, laser eye surgery (LASIK), etc.

A vitreo-retinal surgery is a type of eye surgery that treats problems with the retina or the vitreous. Vitreo-retinal surgery may be performed for treating conditions such as diabetic traction retinal detachment, diabetic vitreous hemorrhage, macular hole, retinal detachment, epimacular membrane, and many other ophthalmic conditions. Cataract surgery involves emulsifying the patient's crystalline lens with an ultrasonic handpiece and aspirating it from the eye. An intraocular lens (IOL) is then implanted in the posterior lens capsule of the eye. During vitreo-retinal, cataract, and other types of surgeries mentioned above and known to one of ordinary skill in the art, various deficiencies may negatively impact the outcome, efficiency, and effectiveness of the surgery and the surgeon's ease of performing the surgery as well as, in certain cases, cause harm to the patient's optical anatomy, etc.

### BRIEF SUMMARY

The present disclosure relates generally to methods and apparatus for performing ophthalmic surgical procedures. In certain embodiments, a method includes ingesting and preparing pre-operative data and intra-operative data associated with a patient's eye for further processing. In certain embodiments, the method further includes integrating the pre-operative data and intra-operative data to generate context sensitive data for further processing. The method also includes classifying and annotating the pre-operative data, the intra-operative data, and the context sensitive data. The method also includes extracting one or more actionable inferences from the pre-operative data, the intra-operative data, context sensitive data, and the classified and annotated data. The method further includes triggering, based on the one or more actionable inferences, one or more actions on an imaging system or a surgical system.

The following description and the related drawings set forth in detail certain illustrative features of one or more embodiments.

### BRIEF DESCRIPTION OF THE DRAWINGS

The appended drawings depict only examples of certain embodiments of the present disclosure and are therefore not to be considered as limiting the scope of this disclosure.

FIG. 1 illustrates an example of a digitally aware system (hereinafter “digital awareness system”) configured with digital awareness technology, according to some embodiments.

FIG. 2 illustrates example operations for use by the digital awareness system of FIG. 1, in accordance with certain embodiments.

FIG. 3 illustrates an example computing device that implements, at least partly, one or more functionalities of the digital awareness system of FIG. 1, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods.

As discussed above, during vitreo-retinal, cataract, and other types of surgeries mentioned above and known to one of ordinary skill in the art, various deficiencies may negatively impact the outcome, efficiency, and effectiveness of the surgery and the surgeon's ease of performing the surgery as well as, in certain cases, cause harm to the patient's optical anatomy, etc. For example, existing surgical systems or platforms (hereinafter “systems”) are technically deficient when it comes to providing surgical image guidance, surgical patient monitoring, virtual assistance, as well as other automated operations that may improve surgical outcomes, reduce the likelihood of physical harm to the patient's eye, and improve the surgery's efficiency and effectiveness, etc. Various types of deficiencies with respect to existing surgical systems are described below.

### Image Guidance Deficiencies

For example, existing surgical systems are not technically capable of identifying toric IOL markings (e.g., three laser dots) to improve lens alignment during IOL implantation as part of cataract surgery. In another example, existing surgical systems are not technically capable of keeping track of incision location during cataract surgery to facilitate easy insertion of an IOL delivery device into the incision for IOL injection purposes. In another example, existing surgical systems are not technically capable of identifying residual lens fragments, after phacoemulsification, to enable efficient removal of crystalline lens material fragments. In yet another example, existing surgical systems are not technically capable of providing image guidance during MIGS (micro-invasive glaucoma surgery) implantation. There are additional examples of image guidance deficiencies associated with existing surgical systems that are omitted for brevity.

### Patient Monitoring Deficiencies

During cataract surgery, surgical instrumentation may rupture the capsular bag and, currently, existing surgical systems are not technically capable of accurately detecting whether or when surgical instrumentation is too close to the capsular bag. In another example, during retina surgery, surgical instrumentation may rupture the retinal tissue and, currently, existing surgical systems are not technically capable of accurately detecting whether or when surgical instrumentation is too close to the retinal tissue. In yet another example, existing surgical systems are not technically capable of evaluating cataract grade to suggest the optical power settings for phacoemulsification. In yet another example, existing surgical systems are not technically capable of monitoring and mapping the path of the vitreous-cutter device (i.e., vitrector) in the eye and suggest region of focus for residual vitreous removal. In another example, existing surgical systems are not technically capable of monitoring the patient's eye condition during the surgery to alert the surgeon about any unexpected conditions. There are additional examples of patient monitoring deficiencies associated with existing surgical systems that are omitted for brevity.

### Virtual Assistance Deficiencies

For example, existing surgical systems are not technically capable of monitoring image quality and adjusting (or recommending to adjust) device settings to enhance viewing. In another example, existing surgical systems are not technically capable of adjusting illumination settings to enhance viewing and detection of ocular features during surgery. In yet another example, existing surgical systems are not technically capable of automating the glide-path of a robotic arm during surgical procedures. There are additional examples of virtual assistance deficiencies associated with existing surgical systems that are omitted for brevity.

### Automation Deficiencies

For example, existing surgical systems are not technically capable of annotating surgical videos for billing and to generate teaching/training aids. In another example, existing surgical systems are not technically capable of automatically generating billing codes for surgeries based on the complexity of the surgery. There are additional examples of automation deficiencies associated with existing surgical systems that are omitted for brevity.

### Digitally Aware Surgical System

The embodiments herein describe a digitally aware surgical system that provides a technical solution to the technical problems and deficiencies described above.

The digitally aware surgical system described herein has at least four key technical capabilities including the (i) capability to analyze data in real time, (ii) capability to process multi-model data (i.e., data that is generated and/or received in different formats simultaneously, such as surgical videos, numerical data, voice data, text, images, signals, etc.), (iii) capability to process data received from a single source or from multiple sources simultaneously (e.g., images captured by a camera, internal sensor data, voice recording from a microphone), and (iv) capability to make inferences using the received and processed data in relation to the status of the surgical procedure, surgical instrumentation, status of the patient or their eye, controlling surgical equipment.

Digital awareness technology, as described herein, can deliver smart functionality for surgical systems. Smart functionality for ocular surgical systems can take multiple forms in the operating room (OR), such as, image guidance based operations, patient monitoring, virtual assistant for the surgeon, and/or service automation. Incorporating the smart functionality, described by the embodiments herein, result in many improvements over existing surgical systems. The improved surgical systems described herein are capable of assisting surgeons in performing surgical tasks with higher accuracy, efficiency, and/or safety, ultimately leading to a better surgical outcome for each patient.

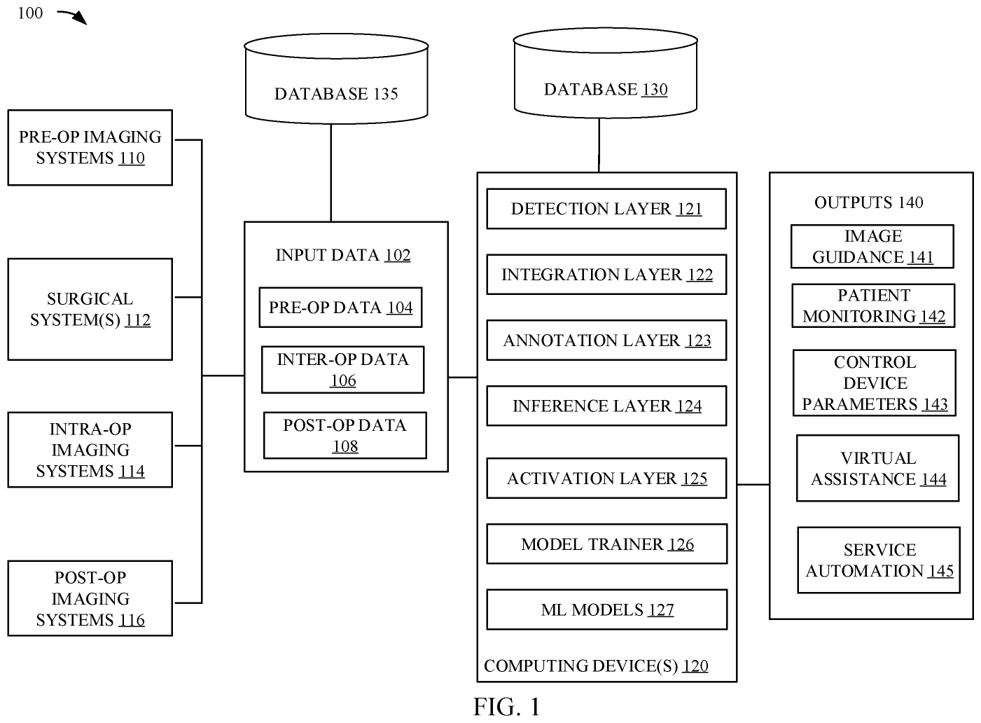

FIG. 1 illustrates an example of digitally aware system 100 (hereinafter “digital awareness system”) configured with digital awareness technology, according to some embodiments. As shown, digital awareness system 100 includes a variety of systems, such as one or more pre-operative (hereinafter “pre-op”) imaging systems 110, one or more surgical systems 112, one or more intra-operative (hereinafter “intra-op”) imaging systems 114, and post-operative (hereinafter “post-op”) imaging systems 116.

Pre-op imaging systems 110, surgical systems 112, intra-op imaging systems 114, and post-op imaging system 116 may be co-located or located in various locations, including diagnostic clinics, surgical clinics, hospitals, and other locations. Whether co-located or located across various locations, systems 110, 112, 114, and 116 may each generate data that can be communicated and used as part of input data 102 over one or more networks (e.g., a local area network, a wide area network, and/or the Internet) to other systems 110, 112, 114, and 116, computing system(s) 120, and/or to databases 130 and 135.

Pre-op imaging systems 110 may refer to any number of diagnostic systems that may be used, prior to surgery, at a clinic for obtaining multi-dimensional images and/or measurements of ophthalmic anatomy such as an optical coherence tomography (OCT) system, a rotating camera (e.g., a Scheimpflug camera), a magnetic resonance imaging (MRI) system, a keratometer, an ophthalmometer, an optical biometer, a topographer, a retinal camera, any type of intra-operative optical measurement system, such as an intra-operative aberrometer, and/or any other type of optical measurement/imaging system. Examples of OCT systems are described in further detail in U.S. Pat. No. 9,618,322 disclosing “Process for Optical Coherence Tomography and Apparatus for Optical Coherence Tomography” and U.S. Pat. App. Pub. No. 2018/0104100 disclosing “Optical Coherence Tomography Cross View Image”, both of which are hereby incorporated by reference in their entirety.

Surgical systems 112, may refer to any number of systems for performing a variety of ophthalmic surgical procedures. As an example, surgical system 112 may include consoles for performing vitreo-retinal surgeries (e.g., Constellation console manufactured by Alcon Inc., Switzerland), cataract surgeries (e.g., Centurion console manufactured by Alcon Inc., Switzerland), and many other systems used for performing a variety of ophthalmic surgeries, as known to one of ordinary skill in the art. Note that, herein, the term “system” is also inclusive of the terms console and device.

Intra-op imaging systems 114 may include any systems that may obtain imaging or video data as well as measurements associated with a patient's eye during a surgical procedure. An example of an intra-operative imaging system 114 used for cataract surgery is the Ora™ with Verifeye™ (Alcon Inc., Switzerland), which is used to provide intra-operative measurements of the eye, including one or more of the curvature of the cornea, axial length of the eye, white-to-white diameter of the cornea, etc. Other types of intra-op systems used for generating and providing intra-op data may include digital microscopes, such as three-dimensional stereoscopic digital microscopes (e.g., NGENUITY® 3D Visualization System (Alcon Inc., Switzerland). A variety of other intra-op imaging systems may also be used, as known to one of ordinary skill in the art.

Post-op imaging systems 116 may refer to any number diagnostic systems that may be used, post-surgery, at a clinic for obtaining multi-dimensional images and/or measurements of ophthalmic anatomy. Post-op imaging systems 116 may be the same as pre-op imaging systems 110, described above.

Input data 102 includes pre-op data 104, intra-op data 106, and post-op data 108. Pre-op data 104 may include information about the patient, including data that may be received from database 135 (e.g., a database, such as an electronic medical record (EMR) database for storing patient history information) and data that is generated and provided by pre-op imaging systems 110 about the patient's eye. For example, pre-op data 104 may include patient history information, including one or more relevant physiological measurements for the patient that are not directly related to the eye, such as one or more of age, height, weight, body mass index, genetic makeup, race, ethnicity, sex, blood pressure, other demographic and health related information, and/or the like. In some examples, the patient history may further include one or more relevant risk factors including smoking history, diabetes, heart disease, other underlying conditions, prior surgeries, and/or the like and/or a family history for one or more of these risk factors.

Data that is generated and provided by pre-op imaging systems 110 about the patient's eye may include one or more pre-op measurements and images as well as any measurements or other types of information extracted from the one or more pre-op images. As an example, pre-op images may include images of one or more optical components of the eye (e.g., retina, vitreous, crystalline lens, cornea, etc.). Pre-op measurements may include the patient's axial length of the eye, corneal curvature, anterior chamber depth, white-to-white diameter of the cornea, lens thickness, effective lens position, as well as measurements relating to retinal diseases and other conditions, as known to one of ordinary skill in the art.

Intra-op data 106 may include any information obtained or generated during or as a result of the patient's surgical procedure. For example, intra-op data 106 may include data inputted into (e.g., by a user), or generated and provided (e.g., automatically) by surgical systems 112 as well as intra-op imaging systems 114, which may be present in an operating room during the patient's surgical procedure. In particular, such intra-op imaging data may include one or more intra-operative images and/or measurements, including images and/or measurements of the eye obtained as the procedure is being performed.

Examples of intra-op data 106 includes surgical videos and images captured by a digital microscope and images captured by a surgical microscope, surgical system data that includes system parameters, active settings, and UI/UX/control status set by a surgeon or the staff, other data modality pertinent to the surgeon who is interacting with the system, such as voice commands, gesture-based commands, or commands that can be received by tracking eye gaze of the surgeon, patient monitoring information, such as a patient eye position obtained by a system other than a surgical microscope, data obtained from sensors embedded in a surgical/imaging system, surgical procedure specific data associated with the patient's optical components, such as the cornea, cataract, vitreoretinal components, MIGS related components (e.g., details pertinent to a cataract procedure including an incision position, IOL types, injector type, illumination settings, etc.).

Post-op data 108 may include one or more post-op measurements and images as well as any measurements or other information extracted from the one or more post-op images. Post-op data 108 may also include patient outcome data, including a post-op satisfaction score. Patient outcome data may also be in relation to treatment efficacy and/or treatment related safety endpoints. Post-op data 108 may be particularly important for algorithm training and to continuously improve the performance of digital awareness system 100.

Computing system(s) 120 may refer to one or more co-located or non-co-located systems that execute layers of instructions shown as detection layer 121, integration layer 122, annotation layer 123, inference layer 124, activation layer 125. Computing system(s) 120 also execute a model trainer 126 as well as one or more machine learning models 127. In certain embodiments, computing system(s) 120 may be cloud-based (e.g., private or public cloud) or located on premises (“on-prem”), or a combination thereof.

In certain embodiments, when there are multiple computing systems 120, different instructions (e.g., instruction layers 121-125, model trainer 126, and ML models 127) may be executed by different computing systems 120. For example, one of the multiple computing systems 120 may be configured to execute detection layer 121 and another one of the multiple computing systems 120 may execute ML models 127. In another example, one of the multiple computing systems 120 may be configured to execute detection layer 121 and another one of the multiple computing systems 120 may be configured to execute integration layer 122. In certain embodiments, one or more instruction layers 121-125, model trainer 126, and ML models 127 may be executed by multiple computing systems 120 in a distributed and decentralized manner. In certain embodiments, one or more of computing systems 120 may be or include one or more of imaging systems 110, 114, and 116, and surgical systems 112 that are used to obtain ophthalmic information or perform ophthalmic surgical procedures, respectively, as described above.

During surgery, instructions layers 121-125 and ML models 127 may be executed to take input data 102 for a specific patient for whom the surgery is being performed and provide certain outputs, such as output 104.

For example, detection layer 121 is configured to ingest input data 102 or any portion thereof and prepare input data for further processing. Integration layer 122 integrates intra-op data 106 with pre-op data 104 to generate context sensitive information for further processing. Annotation layer 123 may be configured to use one or more of the ML models 127 to classify and annotate data generated by detection layer 121 and integration layer 122. Inference layer 124 may be configured with algorithms designed to extract one or more actionable inferences from the data that is generated by detection layer 121, integration layer 122, and annotation layer 123. In other words, data generated by detection layer 121, integration layer 122, and annotation layer 123 is used as input to inference layer 124. Activation layer 125 may be configured with algorithms designed to trigger a set of defined downstream events based on output from inference layer 124. Example outputs of activation layer 125 is shown as outputs 140.

Model trainer 126 includes or refers to one or more AI-based learning algorithms (referred to hereinafter as “AI-based algorithms”) that are configured to use training datasets stored in a database (e.g., database 130) to train ML models 127. Examples of AI-based algorithms are optimization algorithms such as gradient descent, stochastic gradient descent, non-linear conjugate gradient, etc.

In certain embodiments, a trained ML model 127 refers to a function, e.g., with weights and parameters, that can be used by one or more layers 121-125 to make predictions and determinations. A variety of ML models 127 may be trained for and used by different layers 121-125 for different purposes. Example ML models may include different types of neural networks, such as long short-term memory (LSTM) networks, 3D convolutional networks, deep neural networks, or many other types of neural networks or other machine learning models, etc.

Database 130 may refer to a database or storage server configured to store input data 102 associated with each patient as well as training datasets using by model trainer 126 to train ML models 127. Training datasets may include population-based data as well as personalized data.

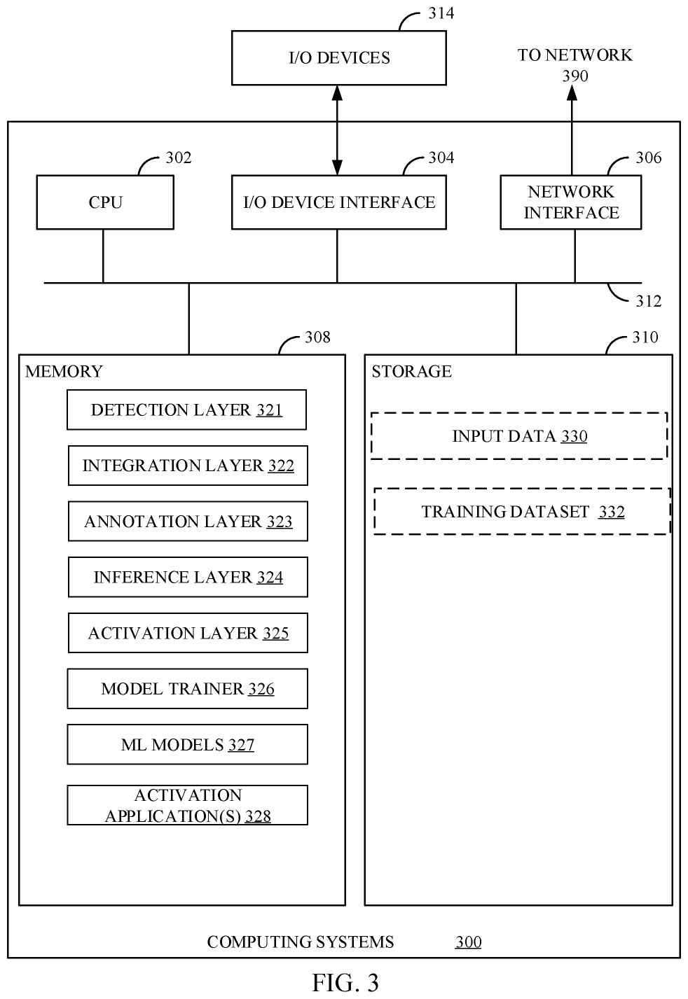

As shown, output 140 is categorized into a number of different outputs, including image guidance 141, patient monitoring 142, control system parameters 143, virtual assistance 144, service automation 145, etc. As described above, outputs 140 may be triggered by computing system(s) 140, such as activation layer 125. Any of the types of outputs 140 discussed above may be provided or caused to be provided by one or more software applications (e.g., activation application(s) 328 of FIG. 3) executing on one or more of imaging systems 110, 114, and, 116 and surgical systems 112.

Image guidance 141 refers to a set of operations provided for guiding a surgical operation. Examples of image guidance based operations include identifying toric IOL (intraocular lens) markings (e.g. three laser dots) on an image to improve lens alignment during implantation in a cataract surgery, keeping track of the incision location during cataract surgery to facilitate easy placement of delivery device for an IOL injection, identifying residual lens fragments after phacoemulsification to enable efficient removal of crystalline lens material fragments, image guidance during MIGS implantation, etc.

Patient monitoring 142 refers to a set of operations performed (e.g., automatically) to monitor aspects of the surgical procedure. Examples of patient monitoring operations include detecting a location of the surgical instrumentation in relation to various tissues or optical components of the eye during cataract procedure to avoid risk of capsular bag rupture, detecting a location of the surgical instrument in relation to various tissues or optical components of the eye during vitreo-retinal procedures to avoid risk of the surgical instrumentation rupturing the retinal tissue, evaluating the cataract grade of the cataract lens during cataract surgery and to suggest an optimal power setting for performing phacoemulsification, monitoring and mapping the path of the vitrectomy cutter device (vitrector) in the eye and suggest region of focus for residual vitreous removal, monitoring the patient's eye condition during the surgery to alert the surgeon about any unexpected conditions, etc.

Control system parameters 143 refer to system parameters that are determined and output by activation layer 125 for reconfiguring and/or controlling/changing the operations of one or more of imaging systems 110, 114, and, 116 and surgical systems 112.

Virtual assistance 144 refers to a set of operations performed to provide virtual assistance to a surgeon including automatically monitoring image quality and suggesting to adjust (or automatically adjusting) system settings to enhance viewing (e.g., suggesting auto-white balance for a 3D visualization system), adjusting illumination settings to enhance viewing and detection of ocular features during surgery, automating the glide-path of a robotic arm during specific surgical procedures, etc.

Service automation 145 refers to a set of operations for automatically providing certain tasks associated with a surgical procedure, including automatically annotating a surgical video for billing and to generate teaching/training aids, automatically generating billing codes for billing based on the complexity of a surgical procedure, automatically processing a surgical video for teaching/training purposes.

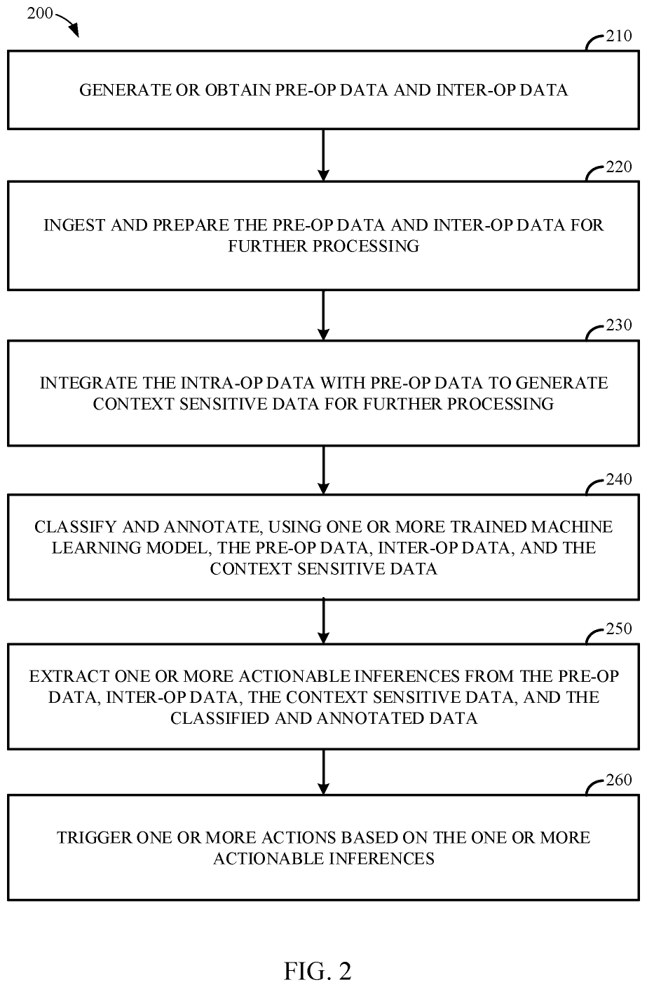

FIG. 2 illustrates operations 200 for use by a digital awareness system (e.g., digital awareness system 100) to provide surgical image guidance, surgical patient monitoring, virtual assistance, as well as other automated operations that may improve surgical outcomes, reduce the likelihood of physical harm to the patient's eye, and improve the surgery's efficiency and effectiveness, according to certain embodiments. Operations 200 may be performed by one or more of computing system(s) 120, one or more of imaging systems 110, 114, and, 116 and surgical systems 112, or any combination thereof.

At operations 210, the digital awareness system generates or obtains pre-op data 104 and intra-op data 106. For example, one or more imaging systems 110 and 114 may generate pre-op data 104 and intra-op data 106, as described above.

At operations 220, the digital awareness system ingests and prepares the pre-op data 104 and intra-op 106 for further processing. For example, operations 220 may be performed by detection layer 121. As an example, detection layer 121 may be configured with one or more machine learning models trained to identify the “toric-dots” on an IOL. In such an example, detection layer 121 may take a raw surgical video feed provided by an intra-op imaging system 114, such as a digital camera, as input, and output an “annotated surgical video” where the “toric IOL dots” are identified and marked. This “annotated surgical video” with the “toric IOL dots” identified and marked may be used for providing image guidance to a surgeon for toric IOL alignment purposes.

In another example, detection layer 121 may be configured with one or more machine learning models trained to identify specific landmarks in the eye that correspond to regions where a MIGS device would need to be placed, based on the mode of action (MoA) of the MIGS device. Identifying these specific landmarks in the eye are particularly useful in MIGS surgery. For example, prior to a surgeon performing the MIGS surgery in an operating room, based on an indication that the surgeon is about to perform a MIGS surgery, detection layer 121 can be configured to (e.g., automatically) perform the task of identifying all the relevant landmarks in the eye and generating an “annotated surgical video” to feed as input to integration layer 122. The annotated surgical video will then be processed and operated on by the additional layers 122-125 to provide image guidance for MIGS surgery.

At operations 230, the digital awareness system integrates the pre-op data with the intra-op data to generate context sensitive data for further processing. In certain embodiments, operations 230 may be performed by integration layer 122. In certain embodiments, integration layer 122 may integrate the pre-op data with the intra-op data by correlating pre-op data with the intra-op based on their corresponding time-stamps.

To continue with the example use-case described above in relation to providing image guidance for MIGS implantation, integration layer 122 may combine two of more outputs from detection layer 121, each output identifying the (i) ocular land-marks, (ii) the MIGS device model that is being implanted, and (iii) the instrumentation being used for the MIGS implantation, to generate a consolidated view of an ocular surgical video image that (i) highlights the ocular landmark appropriate for the given MIGS device model and (ii) overlays the optimal pathway for the MIGS device implantation.

In another example, integration layer 122 may queue pre-op diagnostic images and automatically load them into an intra-op surgical video stream, thereby allowing the surgeon to view the pre-op diagnostic images and the video stream side-by-side intra-operatively during different stages of the surgical procedure. Integration layer 121 may queue and load the pre-op images depending on the surgical stage of the procedure, thereby ensuring that the images are loaded into the right video stream at the right stage of the procedure.

At operations 240, the digital awareness system classifies and annotates, using one or more trained machine learning models, the pre-op data and intra-op data (e.g., received from detection layer 121) and the context sensitive data (e.g., received from integration layer 122). In certain embodiments, operations 240 are performed by annotation layer 123. For example, when dealing with a continuous flow of video data, annotation layer 123 may use a variety of machine learning models (e.g., ML models 127), such as neural networks, to perform feature extraction on the video data and predict the surgical step that is currently occurring or being performed.

For instance, annotation layer 123 may perform feature extraction on each video frame using two-dimensional convolution neutral networks (2D-CNN) such as a visual geometry group (VGG), Inception, or a vision transformer referred to as ViT. Features of each frames are fed to a RNN (recurrent neural network) that handles sequential data to continuously predict the surgical step label (using a RNN, such as unidirectional LSTM). A surgical step label refers to a label that identifies the surgical step being performed in real-time.

In another example, annotation layer 123 may perform 3D feature extraction from each video segments comprising multiple frames by using, e.g., a 3D-CNN. Features of each video segments may then be fed to a dense FC (fully-connected) network to predict the surgical step label.

In yet another example, annotation layer 123 may perform feature extraction from each video frame, such as described above, but instead of feeding the features of each frame to a RNN, the features in each frame may be directly used to predict the surgical step label. Such an approach is simpler than feeding the features of each frame to a RNN but less resource intensive.

At operations 250, the digital awareness system extracts one or more actionable inferences from the pre-op data, intra-op data, the context sensitive data, and the classified and annotated data. In certain embodiments, operations 250 are performed by inference layer 124. For example, inference layer 124 may use one or more ML models (e.g., ML models 127) to make determinations or predictions that may then be used to trigger one or more actions (e.g., by activation layer 125) to provide outputs 140. As an example, the determinations or predictions may include a determination about the distance between an instrument tip and a specific landmark in the patient eye, a determination about image contrast, color, and defocus based on specific image quality metrics, and detection of a change in tasks or surgical steps within an ongoing surgical procedure.

At operations 260, the digital awareness system triggers a set of defined downstream events based on the output of operations 250. In certain embodiments, operations 260 are performed by activation layer 125 based on the output from inference layer 124. As discussed above, the output from activation layer 125 may take many forms, examples of which were provided above as outputs 140 in relation to FIG. 1. Additional examples of actions that may be triggered by activation layer 125 include flashing a color code on a heads-up display of a 3D visualization system (e.g., the NGENUITY system provided by Alcon Inc., Switzerland) based on the inferred proximity of the surgical instrument to specific landmarks in the patient's eye, sending push notifications to a surgeon to accept an updated device display setting to rectify sub-optimal image quality metrics, pushing a log file to document a surgical procedure with representative snapshots, text description, and automatic billing, etc.

FIG. 3 illustrates an example computing system 300 that implements, at least partly, one or more functionalities of a digital awareness system, such as digital awareness system 100. Computing system 300 may be any one of imaging systems 110, 114, 116, surgical systems 112, and computing systems 120 of FIG. 1.

As shown, computing system 300 includes a central processing unit (CPU) 302, one or more I/O device interfaces 304, which may allow for the connection of various I/O devices 314 (e.g., keyboards, displays, mouse devices, pen input, etc.) to computing system 300, network interface 306 through which computing system 300 is connected to network 390 (which may be a local network, an intranet, the internet, or any other group of computing systems communicatively connected to each other, as described in relation to FIG. 1), a memory 308, storage 310, and an interconnect 312.

In cases where computing system 300 is an imaging system (e.g., imaging system 110, 114, or 116), computing system 300 may further include one or more optical components for obtaining ophthalmic imaging of a patient's eye as well as any other components known to one of ordinary skill in the art. In cases where computing system 300 is a surgical system (e.g., surgical systems 112), computing system 300 may further include many other components known to one of ordinary skill in the art to perform the ophthalmic surgeries described above in relation to FIG. 1 and known to one of ordinary skill in the art.

CPU 302 may retrieve and execute programming instructions stored in the memory 308. Similarly, CPU 302 may retrieve and store application data residing in the memory 308. The interconnect 312 transmits programming instructions and application data, among CPU 302, I/O device interface 304, network interface 306, memory 308, and storage 310. CPU 302 is included to be representative of a single CPU, multiple CPUs, a single CPU having multiple processing cores, and the like.

Memory 308 is representative of a volatile memory, such as a random access memory, and/or a nonvolatile memory, such as nonvolatile random access memory, phase change random access memory, or the like. As shown, memory 608 includes detection layer 321, integration layer 322, annotation layer 323, inference layer 324, activation layer 325, model trainer 326, ML models 327, and activation application(s) 328. The functionalities of detection layer 321, integration layer 322, annotation layer 323, inference layer 324, activation layer 325, model trainer 326, ML models 327 are similar or identical to the functionalities of detection layer 121, integration layer 122, annotation layer 123, inference layer 124, activation layer 125, model trainer 126, and ML models 127. Note that all of the instructions, modules, layers, and applications in memory 208 are being shown in dashed boxes to show that they are optional because, depending on the functionality of computing system 300 one or more of the instructions, modules, layers, and applications may be executed by computing system 300 while others may not be. For example, in cases where computing system 300 is an imaging system (e.g., one of imaging systems 110, 114, or 116) or a surgical system (e.g., surgical system 112), memory 308 may, in certain embodiments, store an activation application 328 (in order to trigger one or more actions based on outputs 140) but not model trainer 326. In cases where computing system 300 is a server system (e.g., not an imaging system or surgical system) configured to train ML models 327, memory 308 may, in certain embodiments, store model trainer 326 and not an activation application 328.

Storage 310 may be non-volatile memory, such as a disk drive, solid state drive, or a collection of storage devices distributed across multiple storage systems. Storage 310 may optionally store input data 330 (e.g., similar or identical to input data 102) as well as a training dataset 332. Training dataset 330 may be used by model trainer 326 to train ML models 327 as described above. Training dataset 330 may also be stored in external storage, such as a database (e.g., database 130).

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method of performing ophthalmic surgical procedures, comprising:
ingesting and preparing pre-operative data and intra-operative data associated with a patient's eye for further processing;
integrating the pre-operative data and intra-operative data to generate context sensitive data for further processing;
classifying and annotating the pre-operative data, the intra-operative data, and the context sensitive data;
extracting one or more actionable inferences from the pre-operative data, the intra-operative data, context sensitive data, and the classified and annotated data; and
triggering, based on the one or more actionable inferences, one or more actions on an imaging system or a surgical system.

2. The method of claim 1, wherein the pre-operative data and intra-operative data are generated by one or more ophthalmic imaging systems, the method further comprising:
receiving the pre-operative data and intra-operative data from the one or more ophthalmic imaging systems.

3. The method of claim 1, wherein integrating the pre-operative data and intra-operative data is based on time-stamps associated with the pre-operative data and time-stamps associated with the intra-operative data.

4. The method of claim 1, wherein the classifying and annotating further comprise performing feature extraction on the pre-operative data, the intra-operative data, and the context sensitive data using one or more trained machine learning models.

5. The method of claim 1, wherein the one or more actionable inferences include:
a determination about a distance between an instrument tip and a specific landmark in the patient eye,
a determination about image contrast, color, and defocus based on specific image quality metrics, or
detection of a change in tasks or surgical steps within an ongoing surgical procedure.

6. The method of claim 1, wherein the one or more actions comprise:
providing image guidance;
providing patient monitoring; or
providing virtual assistance.

7. The method of claim 6, wherein providing image guidance comprises flashing a code on a heads-up display of a 3D visualization system based on an inferred proximity of a surgical instrument to a specific landmark in the patient's eye.

8. An ophthalmic imaging or surgical system, comprising:
a memory comprising executable instructions; and
a processor in data communication with the memory and configured to execute the instructions to cause the ophthalmic imaging or surgical system to:
ingest and prepare pre-operative data and intra-operative data associated with a patient's eye for further processing;
integrate the pre-operative data and intra-operative data to generate context sensitive data for further processing;
classify and annotate the pre-operative data, the intra-operative data, and the context sensitive data;
extract one or more actionable inferences from the pre-operative data, the intra-operative data, context sensitive data, and the classified and annotated data; and
perform, based on the one or more actionable inferences, one or more actions at the ophthalmic imaging system or surgical system.

9. The ophthalmic imaging or surgical system of claim 8, wherein the processor is further configured to cause the ophthalmic imaging or surgical system to generate at least part of the intra-operative data.

10. The ophthalmic imaging or surgical system of claim 8, wherein integrating the pre-operative data and intra-operative data is based on time-stamps associated with the pre-operative data and time-stamps associated with the intra-operative data.

11. The ophthalmic imaging or surgical system of claim 8, wherein the classifying and annotating further comprise performing feature extraction on the pre-operative data, the intra-operative data, and the context sensitive data using one or more trained machine learning models.

12. The ophthalmic imaging or surgical system of claim 8, wherein the one or more actionable inferences include:
a determination about a distance between an instrument tip and a specific landmark in the patient eye,
a determination about image contrast, color, and defocus based on specific image quality metrics, or
detection of a change in tasks or surgical steps within an ongoing surgical procedure.

13. The ophthalmic imaging or surgical system of claim 8, wherein the one or more actions comprise:
providing image guidance;
providing patient monitoring; or
providing virtual assistance.

14. The ophthalmic imaging or surgical system of claim 13, wherein providing image guidance comprises flashing a code on a heads-up display of a 3D visualization system based on an inferred proximity of a surgical instrument to specific landmarks in the patient's eye.
